SEQUENCE LISTING (29 sequences)

SEQ ID NO: 1 - DNA, 25 bp, Artificial sequence
  oligonucleotide primer with homology to transgenic corn
agtatccttcgcaagacccttcctc

SEQ ID NO: 2 - DNA, 25 bp, Artificial sequence
  oligonucleotide primer with homology to transgenic corn
gcattcagagaaacgtggcagtaac

SEQ ID NO: 3 - DNA, 25 bp, Artificial sequence
  oligonucleotide primer with homology to transgenic plants
attgatgtgatatctccactgacgt

SEQ ID NO: 4 - DNA, 25 bp, Artificial sequence
  oligonucleotide primer with homology to transgenic plants
cctctccaaatgaaatgaacttcct

SEQ ID NO: 5 - DNA, 21 bp, Artificial sequence
  oligonucleotide primer with homology to transgenic plants
ccacgtcttcaaagcaagtgg

SEQ ID NO: 6 - DNA, 25 bp, Artificial sequence
  oligonucleotide primer with homology to transgenic plants
tcctctccaaatgaaatgaacttcc

SEQ ID NO: 7 - DNA, 25 bp, Artificial sequence
  oligonucleotide primer with homology to transgenic plants
attgatgtgatatctccactgacgt

SEQ ID NO: 8 - DNA, 20 bp, Artificial sequence
  oligonucleotide primer with homology to transgenic plants
ttatcctagtttgcgcgcta

SEQ ID NO: 9 - DNA, 25 bp, Artificial sequence
  oligonucleotide primer with homology to transgenic plants
tatctccactgacgtaagggatgac

SEQ ID NO: 10 - DNA, 25 bp, Artificial sequence
  oligonucleotide primer with homology to transgenic plants
tgccctataacaccaacatgtgctt

SEQ ID NO: 11 - DNA, 25 bp, Artificial sequence
  oligonucleotide primer with homology to transgenic plants
cggatggtccttatgcaattttgtc

SEQ ID NO: 12 - DNA, 22 bp, Artificial sequence
  oligonucleotide primer with homology to transgenic plants
ctctcggcgtagatttggtaca

SEQ ID NO: 13 - DNA, 25 bp, Artificial sequence
  oligonucleotide primer with homology to transgenic plants
agattcttcactccgatgcagccta

SEQ ID NO: 14 - DNA, 22 bp, Artificial sequence
  oligonucleotide primer with homology to transgenic plants
tggacaacaacccaaacatcaa

SEQ ID NO: 15 - DNA, 26 bp, Artificial sequence
  oligonucleotide primer with homology to transgenic plants
tggattttggttttaggaattagaaa

SEQ ID NO: 16 - DNA, 22 bp, Artificial sequence
  oligonucleotide primer with homology to transgenic plants
ggacaacaaccacaacatcaac

SEQ ID NO: 17 - DNA, 19 bp, Artificial sequence
  oligonucleotide primer with homology to transgenic plants
cgatgggggtgtaaccggt

SEQ ID NO: 18 - DNA, 19 bp, Artificial sequence
  oligonucleotide primer with homology to transgenic plants
gcacgaactcgctaagcag

SEQ ID NO: 19 - DNA, 18 bp, Artificial sequence
  oligonucleotide primer with homology to transgenic plants
cggccccgagttcacctt

SEQ ID NO: 20 - DNA, 23 bp, Artificial sequence
  oligonucleotide primer with homology to transgenic plants
ctgctggggatgatgttgttcmg

SEQ ID NO: 21 - DNA, 25 bp, Artificial sequence
  oligonucleotide primer with homology to transgenic plants
cactacaaatgccatcattgcgata

SEQ ID NO: 22 - DNA, 25 bp, Artificial sequence
  oligonucleotide primer with homology to transgenic plants
agatcatcaatccactcttgtggtg

SEQ ID NO: 23 - DNA, 18 bp, Artificial sequence
  oligonucleotide primer with homology to transgenic plants
gatagcgctcccgcagac

SEQ ID NO: 24 - DNA, 19 bp, Artificial sequence
  oligonucleotide primer with homology to transgenic plants
gcactgaatttgtgaaccc

SEQ ID NO: 25 - DNA, 21 bp, Artificial sequence
  oligonucleotide primer with homology to transgenic plants
ctatattttgttttctatcgc

SEQ ID NO: 26 - DNA, 23 bp, Artificial sequence
  oligonucleotide primer with homology to transgenic plants
catcgtcaaccactacatcgaga

SEQ ID NO: 27 - DNA, 18 bp, Artificial sequence
  oligonucleotide primer with homology to transgenic plants
gatagcgctcccgcagac

SEQ ID NO: 28 - DNA, 20 bp, Artificial sequence
  oligonucleotide primer with homology to transgenic plants
actgggctccacgctctaca

SEQ ID NO: 29 - DNA, 21 bp, Artificial sequence
  oligonucleotide primer with homology to transgenic plants
aaacccacgtcatgccagttc

(19) United States
(12) Patent Application Publication
(10) Pub. No.: US 2007/0077561 A1
(43) Pub. Date: Apr. 5, 2007

(54) Detection of transgenes of genetically modified organisms using pyro luminescence

(76) Inventor: Yan Hong, Singapore (SG)

Correspondence Address: ROTHWELL, FIGG, ERNST & MANBECK, P.C., 1425 K STREET, N.W., SUITE 800, WASHINGTON, DC, 20005, US

(21) Appl. No.: 10/552,660

(22) PCT Filed: Apr. 14, 2004
(86) PCT No.: PCT/SG04/00093

§ 371 Date: Oct. 11, 2005

Related U.S. Application Data
(60) Provisional application No. 60/462,308, filed on Apr. 14, 2003.

(51) Int. Cl. C12Q 1/68; C12N 9/22

(52) U.S. Cl. 435/6; 435/199

(57) ABSTRACT

The present application provides a method for identifying the presence of a transgene of a genetically modified organism in a sample wherein the nucleic acid sequence of the transgene is replicated and detected as the release of PPi from the deoxynucleotide triphosphate precursors consumed in the replication reaction. This pyro luminescent detection system can be applied qualitatively or quantitatively to detect the presence of a specific transgene in a target DNA sample such as sample containing DNA from a genetically modified organism.

### CROSS REFERENCE TO RELATED APPLICATIONS

This is a Non-Provisional U.S. National Phase Application of PCT Application PCT/SG2004/000093, filed Apr. 14, 2004, which claims benefit of U.S. Provisional Application 60/462,308, which was filed on Apr. 14, 2003.

The present invention is directed to methods for the detection and/or quantification of a nucleic acid in a sample. More specifically, the invention is directed to detecting a nucleic acid in a sample by using the nucleic acid as a template for nucleic acid replication and detecting the release of pyrophosphate (PPi) during the replication process.

The publications and other materials used herein to illuminate the background of the invention or provide additional details respecting the practice, are incorporated by reference.

### BACKGROUND OF THE INVENTION

In order to detect the presence of a specific nucleic acid in a sample, it is generally required that large amounts of the specific sequence be present so that method such as gel electrophoresis and/or probe-specific hybridization can be performed. For qualitative purposes, amplified fragments are often analyzed by agarose gel electrophoresis. Quantitation of a transgene can be accomplished, for example, through real time polymerase chain reaction (PCR), wherein an amplified product is detected as it is made. The presence of transgenes introduced by genetic modification (GM) is mainly detected through PCR reactions to detect the presence of a specific nucleic acid in a sample encoding the transgene. Such PCR reactions depend on binding of two short oligonucleotides to specific regions on template DNA followed by polymerase chain reaction to amplify specific DNA fragments. Nucleic acid amplification methods, such as polymerase chain reaction (PCR), ligase chain reaction (LCR), nucleic acid sequence based amplification (NASBA) and related techniques have become a central technology in genetic engineering and molecular biology. These methods enable the detection of single copy genes and/or DNA fragments with a very high degree of sensitivity, due to the extreme amplification of the signal obtained. As a result, these methods are gradually becoming critical methods in genetic diagnostics in addition to methods in research oriented environments.

In an effort to eliminate the need for sample transfers, so called “homogeneous” or “real-time” methods for the detection of PCR amplified products have been developed. One of the key advantages of real time PCR is the ability to include all necessary components for both amplification and detection in a homogenous reaction system. While several schemes have been put forward for detecting specific nucleic acids in homogeneous solutions (see, e.g., Heller et al. European Patent Application 82303699.1, Morrison et al. (1989) Anal. Biochem., 183: 231-244, Cardullo et al. (1988) Proc. Natl. Acad. Sci. USA, 85: 8790-8794, Morrison et al. (1993) Biochem., 32: 3095-3104, and Sixou et al. (1994) Nucl. Acids Res., 22: 662-668), these methods are typically unsuitable for real-time measurements.

Important design goals for real time monitoring methods and instruments include the ability to minimize sample-to-sample variability in multi-sample thermal cycling, automation of pre- and post-reaction processing steps, high speed temperature cycling, minimization of sample volumes, real time measurement of amplification products, minimization of cross contamination, for example, due to “sample carryover” and simplification of equipment required to carry out the process.

U.S. Pat. No. 5,210,015, issued to Gelfand et al., proposes fluorescence-based approaches to provide real time measurements of amplification products during PCR. Such approaches have either employed intercalating dyes (such as ethidium bromide) to indicate the amount of double-stranded DNA present, or they have employed probes containing fluorescence-quencher pairs (also referred to as the “Taq-Man” approach) where the probe is cleaved during amplification to release a fluorescent molecule whose concentration is proportional to the amount of double-stranded DNA present. During amplification, the probe is digested by the nuclease activity of a polymerase when hybridized to the target sequence to cause the fluorescent molecule to be separated from the quencher molecule, thereby causing fluorescence from the reporter molecule to appear.

Other nucleic acid hybridization probe assays utilizing fluorescence resonance energy transfer pairs include those described by Tyagi et al. in U.S. Pat. No. 5,925,517, which utilizes labeled oligonucleotide probes, which we have come to refer to as “Molecular Beacons.” A Molecular Beacon probe is an oligonucleotide whose end regions hybridize with one another to form a “hairpin” in the absence of a target but are separated if the central portion of the probe hybridizes to its target sequence. Non-FRET fluorescent versions of Molecular Beacon-type probes have also been described (See, e.g., U.S. Pat. No. 6,150,097 issued to Tyagi et al.)

These fluorescent detection approaches in general require a fluorescent compound and a second fluorophore or a quencher. Most fluorescent compounds, however, generally suffer the disadvantage that the fluorescent complexes and their binding moieties are relatively large. In addition, the presence of the fluorescer/quencher markers or the nature of the hairpin loop can alter the interaction of the labeled nucleic acid with other molecules either through chemical interactions or through steric hindrance.

Numerous other methods have been disclosed which enable detection and quantitation of nucleic acid polymerization products. A self-sustained sequence replication electrochemiluminescent nucleic acid assay is disclosed in patent application WO 94-US10732 940921 (Kenten, et al.). Hybridization protection assays have also been described. U.S. Pat. No. 5,593,867, issued to Walker, et al., discloses a fluorescence polarization detection of nucleic acid amplification using fluorescently labeled oligonucleotide probes and detector-probe extension products. U.S. Pat. No. 5,340,716, issued to Ullman et al., describes an assay method utilizing photoactivated chemiluminescent labels. Photoactivatable chemiluminescent matrices have also been described, as have assay methods utilizing induced luminescence as in U.S. Pat. No.6,251,581, issued to Ullman et al. U.S. Pat. No. 5,017,473 issued to Wagner discloses a homogeneous chemiluminescence immunoassay using a light absorbing material.

U.S. Pat. No. 6,159,693 discloses methods for detecting specific nucleic acid sequences wherein DNA or RNA probes are hybridized to target nucleic acid sequences. Probes that are complementary to the target sequence at each base are depolymerized. The nucleic acid detection systems utilize the pyrophosphorolysis reaction catalyzed by various polymerases to produce deoxyribonucleoside triphosphates or ribonucleoside triphosphates. dNTPs are transformed to ATP by the action of NDPK. The ATP produced by these reactions is detected by luciferase or NADH based detection systems.

Methods for sequencing a target nucleic acid by detecting the sequential release of PPi's from dNTP precursors during the sequencing reaction have also been described. U.S. Pat. No. 6,210,891, issued to Nyren et al., discloses a method for determining a nucleotide sequence which comprises a method wherein different deoxynucleotides or dideoxy nucleotides are either added to different aliquots of a template-primer mixture or are added successively to the same mixture and wherein the incorporation of a particular base is determined by detecting the PPi released in the sequencing reaction. U.S. Pat. No. 6,258,568, issued to Nyren et al., discloses a method for determining a nucleotide sequence wherein different deoxynucleotides or dideoxy nucleotides are either added to different aliquots of a template-primer mixture or are added successively to the same mixture, wherein the incorporation of a particular base is determined by detecting the PPi released in the sequencing reaction and which further employs a nucleotide-degrading enzyme to cleave unincorporated nucleotides.

The present invention provides a modification of PPi detection during a polymerase reaction that can be used to detect the presence of a defined sequence in a sample, where PPi release is detected during the replication of a target nucleic acid.

### SUMMARY OF THE INVENTION

The present invention provides new methods for the detection of a target nucleic acid in a sample and kits for carrying out methods. The method comprises replicating a new nucleic acid using the target nucleic acid in the sample as template and detecting the consumption of nucleotide triphosphate precursors as the template is replicated. The consumption of nucleotide triphosphate precursors is detected as the release of PPi from the nucleotide triphosphate precursor that is used to synthesize the new nucleic acid from the template. The released PPi is enzymatically converted to ATP in the presence of APS and ATP sulfurylase. The ATP is then reacted with Luciferin in the presence of oxygen to generate light in the form of luminesence.

Thus, the subject matter of the present application provides methods of detecting the presence, absence, or quantity of a target nucleic acid in a sample by replicating a target nucleic acid in the sample and detecting the amount of PPi generated during the replication process. The replication process can be a linear or a logarithmic replication process. In a preferred embodiment, the replication involves a logarithmic replication (i.e., amplification) of the target sequence.

In one aspect, the method as described above is applied to allele-specific amplification. In this application, the nucleic acid template strand is a sense or antisense strand of one allele and is present in admixture. The oligonucleotide primer is complimentary to the specific allele to be amplified. As a result, the desired nucleic acid strand synthesized on the template strand is amplified preferentially over any other nucleic acid.

This invention also provides nucleic acid amplification mixtures comprising a polymerase; detection enzymes for identifying pyrophosphate release; deoxynucleotides, or optionally deoxynucleotide analogues, optionally including, in place of dATP, a dATP analogue which is capable of acting as a substrate for a polymerase but incapable of acting as a substrate for a said PPi-detection enzymes; and optionally a target specific oligonucleotide primer which hybridizes to a target DNA and facilitates polymerase-directed replication of the target sequence.

This invention also provides kits for performing nucleic acid replication or amplification or for detecting the presence absence or quantity of a nucleic acid in a sample. The kits comprise a container containing one or more enzymes, buffers, and/or any of the other reagents useful for practicing the methods of the present invention. An oligonucleotide can be immobilized as an individual dot on a two dimensional solid support, thus allowing all the amplification reactions to be processed in parallel.

### DETAILED DESCRIPTION OF THE INVENTION

The method of the present application can be used to amplify either RNA or DNA. When used to amplify DNA, the nucleic acid polymerase is a DNA polymerase. Amplification can be linear or exponential. Linear amplification is obtained when the target specific primer is the only complementary oligonucleotide used. Exponential amplification is obtained when a second oligonucleotide is present that is complementary to the target nucleic acid strand. Two primers flank the region that is targeted for amplification. RNA can be reverse transcribed into cDNA and then replicated in a linear or logarithmic fashion. When RNA is the initial template, reverse transcriptase is the initial polymerase. A number of methods for enzymatic nucleic acid amplification in vitro have been developed and can be adapted to detect known sequence variants. These include, for example, polymerase chain reaction (Saiki et al., Science 230, 1350-1354 (1985)), ligase chain reaction (Landegrenet al., Science 241, 1077-1080 (1988)), rolling circle amplification (Lizardi et al., Nature Genetics 19, 225-232 (1998)) and other methods as known in the art.

Pyro luminescence detection is a technique based on the detection of the release of pyrophosphate (PPi) from nucleotide triphosphate precursors during template dependent nucleic acid synthesis.

PPi can be determined by many different methods and a number of enzymatic methods have been described in the literature (Reeves et al., (1969), Anal. Biochem., 28, 282-287; Guillory et al., (1971), Anal. Biochem., 39, 170-180; Johnson et al., (1968), Anal. Biochem., 15, 273; Cook et al., (1978), Anal. Biochem. 91, 557-565; and Drake et al., (1979), Anal. Biochem. 94, 117-120). It is preferred in the present methods to use luciferase and luciferin in combination to identify the release of pyrophosphate since the amount of light generated is substantially proportional to the amount of pyrophosphate released which, in turn, is directly proportional to the amount of base incorporated. A method for continuous monitoring of PPi release based on the enzymes ATP sulphurylase and luciferase has been developed by Nyren and Lundin (Anal. Biochem., 151, 504-509, 1985). The method of Nyren may be modified, for example by the use of a more thermostable luciferase and/or ATP sulfurylase. Thus, in a preferred embodiment, luciferase and luciferin are used in combination to identify the release of pyrophosphate since the amount of light generated is substantially proportional to the amount of pyrophosphate released which, in turn, is directly proportional to the amount of base incorporated. The amount of light can readily be determined by a light sensitive device such as, e.g., a luminometer.

The preferred detection enzymes involved in the PPi detection reaction are ATP sulphurylase and Luciferase. In a cascade of enzymatic reactions, visible light is generated that is proportional to the number of incorporated nucleotides, as illustrated below, with the required enzyme for each step indicated in parentheses:
(NA)ₙ+Nucleotide (polymerase)→(NA)ₙ₊₁+PPi
PPi+APS (ATP sulfurylase)→ATP+SO₄²⁻
ATP+Luciferin+O₂(Luciferase)→AMP+PPi+Oxyluciferin+CO₂+light.

The pyro luminescent detection system can be applied to quantitatively detect the presence of a transgene in a target DNA sample. Within a time window, the amount of PPi produced is proportional to the quantity of primer: template nucleic acid for the polymerization reaction. Thus, detection can be conducted after various cycles of PCR so that the method can also be qualitative. Due to the high sensitivity and broad linear range of such a system, it is possible that just a few or even one polymerization reaction without the need for amplification cycling will yield sufficient signal for quantitation. If only one reaction is required, there is no need for a PCR machine, since the polymerization product can be directly detected with, for example a luminometer, following conversion of the PPi. The invention thus provides a method for detecting a genetically modified organism (GMO) which greatly simplifies GMO detection, cuts down operating time and lowers operating costs. Furthermore, detection of PPi with simple enzymatic reactions and luminsecence requires less skill when compared to other techniques of homogenous nucleic acid amplification and detection.

The present invention also contemplates the use of two or three PCR primers to amplify a specific target transgene or nucleic acid to ensure accurate results. The method of the invention may be performed in two steps, but in one embodiment it is performed in a single homogenous reaction in a single tube or environment. When being performed sequentially, a polymerase reaction or a primer extension step is performed, wherein the various nucleotide(s) are incorporated, followed by a detection step, wherein the release of PPi is monitored or detected, to detect whether or not nucleotide incorporation has taken place. Thus, after the polymerase reaction has taken place, samples from the polymerase reaction mix may be removed and analyzed by adding an aliquot of the sample to a reaction mixture containing the luminescence detection enzymes and reactants. Since the preferred detection and quantification is based on a luminometric reaction, this can be easily followed spectrophotometrically. The use of luminometers is well known in the art and described in the literature.

Primers can be designed for any known transgene and for new genes to be introduced into the genome of a subject of interest for the purpose of modifying the genetic makeup of that subject. In one embodiment primers are designed for transgenes that have been introduced into plants. Sample DNAs can be tested with these primers simultaneously and conveniently in microtiter plates. The test DNA may also be spotted onto a solid phase such as a membrane, glass slide or other solid support before primers are added and a detection or amplification reaction is carried out. Alternatively, the primers may be attached to the solid phase material. Each approach allows high throughput detection of the transgene of interest.

The detection methods of the present invention do not require DNA samples that are high in purity. DNAs from processed food are usually of low quality as indicated by smears of small sized genomic DNA, which represent degraded DNA templates. Even if binding of specific PCR primers to template DNA do not result in the synthesis of full length amplified products from a defined template, partial replication of a target and the detection of any PPi release can still be detected. Thus, the pyro luminescent methods of detection described herein have the same level of specificity, but without the requirement of sequence integrity between priming sites for regular PCR detection. These methods would thus be particularly useful in the detection of GMO testing in highly processed foodsamples, for example, and other samples where degradation may be an issue.

The detection methods may also be used in the detection of pathogens in edible food products, cosmetics, medical fluids such as blood and IV solutions, and other products. Primers specific for DNA sequences specific for known pathogens can be designed and used in the detection and amplification methods of the present application to identify contaminated lots of food product, cosmetics or medical fluids.

To carry out the method of the invention, the detection enzymes can be included in the polymerase reaction step ie., in the chain extension reaction step. Thus, the detection enzymes are added to the reaction mix for the polymerase step prior to, simultaneously with or during the polymerase reaction. The reaction mix for the replication step will thus include the four deoxynucleotide triphosphate precursors needed for the reaction (dATP, dTTP, dGTP and dCTP), polymerase, at least one oligonucleotide primer which can act as a template for polymerase-directed replication, and in a preferred embodiment luciferin, APS, ATP suphurylase and luciferase. The polymerase reaction may be initiated by addition of the polymerase or the oligonucleotide primer. The detection enzymes can already be present at the time the reaction is initiated or they may be added with the reagent that initiates the reaction.

ATP can sometimes be present in the reaction mixture during or after replication of a template, for example as an impurity or as a contaminant of dATP added as dNTP precursor. Endogenous ATP may also interfere in the pyrophosphate luciferin system and give an incorrect luminescence reading. However, the endogenous ATP can be removed from a sample by contacting the sample with an immobilized enzyme such as a pyrase that converts ATP into a product which is no longer a substrate for luciferase. See e.g., U.S. Pat. No. 6,258,568. Thus, in one embodiment, replication of the target sequence is carried out using a dATP analogue which does not interfere in the enzymatic PPi detection reaction but which nonetheless may be normally incorporated into a growing DNA chain by a polymerase. See, e.g., U.S. Pat. No. 6,258,568.

The methods of the invention may be performed on nucleic acids attached to a solid support. Methods for detecting specific nucleic acid sequences generally involve immobilization of the target nucleic acid on a solid support such as nitrocellulose paper, cellulose paper, diazotized paper, or a nylon membrane.

The solid support may also be, e.g., a microtiter well or a bead. In general, any solid support may conveniently be used including any of the vast number described in the art, e.g., for separation/immobilization reactions or solid phase assays, such as for example supports comprising particles, fibers or capillaries made, for example, of agarose, cellulose, alginate, Teflon or polystyrene. Magnetic particles are also useful since they can be readily isolated from a reaction mixture yet have superior reaction kinetics over many other forms of support. The solid support may carry functional groups such as hydroxyl, carboxyl, aldehyde or amino groups, or other moieties such as avidin or streptavidin, for the attachment of primers.

The polymerase reaction in each aliquot in the presence of the extension primer and a deoxynucleotide is carried out using a polymerase which will incorporate deoxynucleotides, e.g., T7 polymerase, Klenow or Sequenase Ver. 2.0 (USB U.S.A.). However, it is known that many polymerases have a proof-reading or error checking ability which employs digestion of a growing strand which can result in a significant level of background noise when detecting PPi release. Thus, in preferred embodiments a nonproof-reading polymerase may be used or fluoride ions or nucleotide monophosphates which suppress 3′ digestion by polymerase can be added to the replication reaction.

The reagents, including enzymes, which may be utilized in the polymerized chain reaction (PCR) are known in the art. See, for example, U.S. Pat. Nos. 5,210,015; 4,683,195; 4,683,202; 4,965,188; 4,800,159; and 4,889,818, the relevant portions of which are incorporated by reference. However, preferred cycling conditions for this reaction are about 1 to 30 cycles, more preferably 5 to 20 cycles, and still more preferably 10 to 15 cycles, at alternating between temperatures of about (1) 58° C. to about 95° C. to (2) about 58 to about 95° C. Some of the more preferred temperature combinations being about 95° C. about 58° C., about 58° C., about 74° C., and about 74° C. However, other combinations are also suitable.

A large variety of microorganisms are involved in the contamination of food, cosmetics, medical supplies and fluids, blood products, intravenous solutions, and the like, among other commercial products. In addition, detection of transgenes is often desirable for the detection of unwanted or contaminated bioengineered food products. The methods of the present invention can be utilized in both the detection of unwanted contaminating microorganisms or unwanted contaminating bioengineered foodstuffs. The methods can also be utilized in the many steps required for the preparation, identification and propagation of new plant lines having an inserted transgene.

The nucleic acids detected with the methods of the invention can include fragments thereof, from any source in purified or unpurified form including DNA and RNA, including t-RNA, m-RNA, r-RNA, mitochondrial DNA and RNA, chloroplast DNA and RNA, DNA-RNA hybrids, or mixtures thereof. The nucleic acids can further comprise all or part of genes, chromosomes, plasmids, the genomes of biological material such as microorganisms, e.g., bacteria, yeasts, viruses, viroids, molds, fungi, plants, animals, humans, and the like. The nucleic acids can be only a minor fraction of a complex mixture such as a biological sample and can be obtained from various biological materials by procedures well known in the art.

The oligonucleotide primers utilized in the methods of the invention can comprise in one embodiment a synthetic nucleotide that is single stranded, containing a sequence at its 3′-end that is capable of hybridizing with a defined sequence of the target polynucleotide. Normally, an oligonucleotide primer has at least 80%, preferably 90%, more preferably 95%, most preferably 100%, complementarity to a defined sequence or primer binding site. The number of nucleotides in the hybridizable sequence of an oligonucleotide primer should be such that stringency conditions used to hybridize the oligonucleotide primer will prevent excessive random non-specific hybridization. The oligonucleotide primer generally will be the same as the defined sequence of the target polynucleotide, that is, generally from about 10 to 200 nucleotides in length, preferably from 20 to 50 nucleotides in length.

The term “nucleoside triphosphates” also includes derivatives and analogs thereof, which are exemplified by those derivatives that are recognized in a similar manner to the underivatized nucleoside triphosphates. Examples of such derivatives or analogs, by way of illustration and not limitation, are those which are biotinylated, amine modified, alkylated, and the like and also include phosphorothioate, phosphite, ring atom modified derivatives, and the like.

Nucleotide polymerases as used in the invention comprise a catalyst, usually an enzyme, for forming an extension of a complementary polynucleotide along a DNA or RNA template. The nucleotide polymerase is a template dependent polynucleotide polymerase and utilizes nucleoside triphosphates as building blocks for extending the 3′-end of a polynucleotide to provide a sequence complementary with the polynucleotide template. Usually, the catalysts are enzymes, such as DNA polymerases, for example, prokaryotic DNA polymerase (I, II, or III), T4 DNA polymerase, T7 DNA polymerase, Klenow fragment, reverse transcriptase, Vent DNA polymerase, Pfu DNA polymerase, Taq DNA polymerase, and the like.

The following Examples illustrate the invention. The figures and the sequence listing referred to in the Examples are as follows:

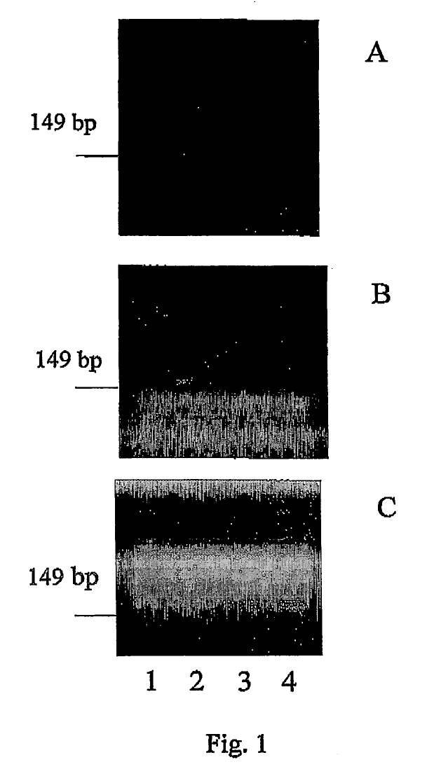

FIG. 1: Photographs of agarose gels analyzing products of a polymerase chain reaction using the pyro luminescence detection method. Panel A shows the results following 10 cycles of PCR; Panel B shows results following 20 cycles of PCT; and Panel C shows results following 30 cycles of PCR. Lane 1, DNA isolated from a maize sample containing 4% of the GMO MON810 maize; Lane 2, 149 basepair (bp) DNA fragment positive control template; Lane 3, negative control containing dATP, but no template DNA and; Lane 4, negative control containing no template DNA, and substituting the inactive dATP analog dATPαS for dATP.

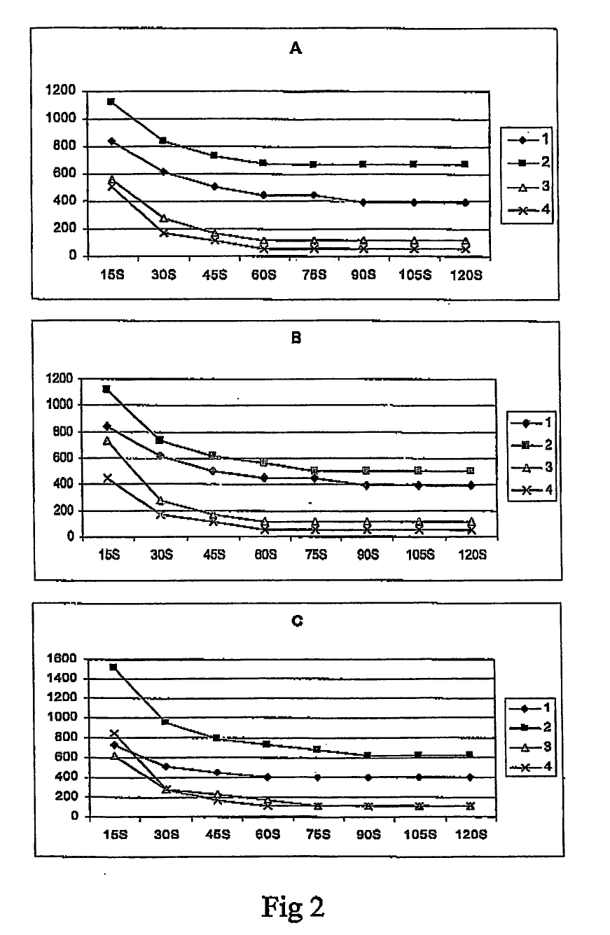

FIG. 2. Fluorescence measurements of samples following PCR. Panel A, 10 cycles of PCR; Panel B, 20 cycles of PR; Panel C, 30 cycles of PCR. Samples are as follows: -♦-, DNA from maize sample containing 4% of the GMO MON810; -▪-, cDNA positive control; -▴-, auto fluorescence control with dATP; -X-, auto fluorescence control with dATPαS.

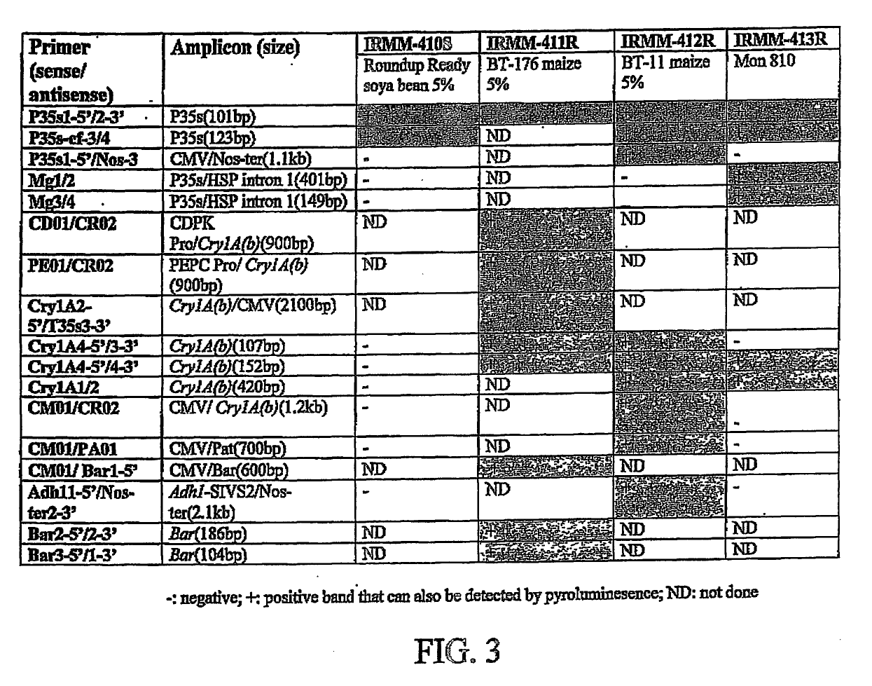

FIG. 3. Results of assays of soybean and maize material containing 5% GMOs with transgenes for resistance to the herbicides glyphosate (Roundup Ready soybeans having an EPSPS gene) and glufosinate (maize having the bar gene) and insect resistance (Bt-176 maize, Bt-11 maize and MON810 maize.)

SEQ ID No. 1—Event specific primer MG3 corresponding to the junction region of the 35S promoter and HSP intron 1 of the transgene inserted into GMO MON810 corn.

SEQ ID No. 2—Event specific primer MG4 corresponding to the junction region of the 35S promoter and HSP intron 1 of the transgene inserted into GMO MON810 corn.

SEQ ID Nos. 3-29—Primer pairs which may be used to detect and/or amplify known transgenes in plants. `

### EXAMPLE 1

### Detection of the Event Specific Target Nucleic Acid of MON810 Maize

Reference samples of maize containing the transgenic GMO corn line MON810 were obtained from Sigma Aldrich Chemical Company. Genomic DNA was isolated from the maize sample using the High Pure GMO sample preparation kit available from Roche Diagnostics Corporation of Indianapolis, Ind. A pair of event specific primers, MG3:5′-agt atc ctt cgc aag acc ctt cct c-3′(SEQ ID NO. 1) and MG4:5′-gca ttc aga gaa acg tgg cag taa c-3′(SEQ ID NO. 2) were used to amplify a 149 basepair fragment of the transgene corresponding to the region of the junction of 35S promoter and HSP intron 1 of MON810 maize.

PCR REACTION: PCR was performed using the PTC-100 thermal cycler (available from MJ Research, Inc). The reaction was carried out in a total volume of 30 μl and contained 0.2 μl of each of the primers MG3 and MG4, 0.25 mM of each of the four dNTPs (dATP, dCTP, dGTP, dTTP), 1.5 mM MgCl₂, 1.5 units of Taq DNA polymerase and 1 μl (10 ng-100 ng) template genomic DNA. The Taq DNA polymerase was from Qiagen.

The thermal cycler program consisted of the following steps: a denaturation step of 4 minutes at 94° C.; cycling steps consisting of a 45 second incubation at 94° C., a 30 second incubation at 60° C., and a 45 second incubation at 72° C.; and an extension step of 7 minutes at 72° C. The reactions were conducted in three sets and allowed to run for 10, 20 and 30 cycles respectively.

FLUORESCENCE DETECTION: Autofluorescence was carried out by pyro luminescence, a technique that detects the release of pyrophosphate (PPi) from nucleotide triphosphates during template dependent nucleic acid synthesis by means of a Luciferase-Luciferin-based reaction. The reaction was carried out in a buffer containing 0.1M Tris-acetate, 2 mM EDTA, 10 mM Magnesium acetate, 0.1% bovine serum albumin, 1 mM dithiothreitol, 0.4 mg/ml polyvinylpyrrolidone, 100 μg/ml Luciferin, 0.3 units/ml ATP sulfurylase, 5 μM Adenosine 5′-phosphosulfate and 20 ng Luciferase. (The Luciferin, Luciferase and ATP sulfurylase were obtained from Sigma Aldrich Chemical Company.) A 5 μl sample of the PCR reaction products was mixed with 100 μl of fresh autofluorescence detection buffer immediately before fluorescence readings were taken. The readings were taken at 15 second-intervals for 2 minutes in a VersaFluor Fluorometer (available from Bio-Rad Laboratories). Deionized water was used as the blank.

RESULTS: Event specific target DNA in genomic DNA isolated from a reference sample which contains 4% of the GMO MON810 maize was detected and amplified by PCR. The products of the PCR were analyzed by agarose gel electrophoresis, as shown in FIG. 1 and by fluorescence detection, as shown in FIG. 2. Ten μl of the PCR products were separated on a 2% agarose gel in 1×TAE buffer. (The gel also contained a 100-bp molecular marker available from New England Biolabs.)

A specific 149 bp DNA fragment of the transgene inserted into MON810 corn was amplified (data not shown) using the two event specific primers MG3 (SEQ ID NO:1) and MG4 (SEQ ID NO:2). The 149 bp fragment was gel purified and used as the positive control template for the PCR data presented in FIG. 1. The PCR amplification product of this fragment is shown in lanes 2 of panels A, B and C of FIG. 1. Corresponding autofluorescence readings are presented in FIG. 2 (-▪-).

The PCR product of the reaction having maize genomic DNA is presented in Lane 1 of FIG. 1 and indicated by -♦- in FIG. 2. Negative control reactions having no template DNA were also carried to determine if the presence of dATP in PCR reaction creates a background fluorescence too high to allow detection of amplified DNA product. The results of this reaction are shown in lane 3 of panels A, B and C of FIG. 1 and the fluorescence measurements of the corresponding panels A, B and C of FIG. 2. (-▴-). A second negative control having no template DNA, and including the inactive dATP analog dATPαS at a final concentration of 0.25 mM rather than dATP, was done to evaluate the background fluorescence levels produced by the dATP in the PCR reactions.

The results demonstrate that the autofluorescence level is high for the initial 15 seconds of the reaction, but it gradually stabilizes between 30 seconds and 2 minutes to provide a workable window during which reliable autofluorescence measurements can be made.

A 149 bp DNA fragment is detectable on the agarose gel for the positive control (Panel A, lane 2) after ten cycles of PCR at an autofluorescence level much higher than two negative controls having no template DNA. Moreover, the two negative controls with dATP and dATPαS have comparable levels of autofluorescence, suggesting that the carryover of dATP from the PCR reaction does not seriously increase autofluorescence background. The use of dATPαS in the reaction does not substantially effect the levels of autofluorescense obtained.

In the sample containing the genomic DNA from maize material comprising 4% of the GMO MON810, the specific 149 bp fragment amplified by primers MG3 and MG4 only becomes detectable after 30 cycles of PCR. Background levels of autofluorescence, in contrast, are detectable after only 10 cycles of PCR. This result suggests that better sensitivities will be obtained by carrying out PCR for multiple cycles as one would do for quantitative PCR, than by carrying out fewer cycles as one would do for qualitative PCR.

The present results demonstrate that detection of pyrophosphate levels by Luciferin autofluorescence in a sample following a PCR reaction can be used to detect the presence of the transgenes of GMOs. In this method, the carryover of a small amount of dATP from the PCR reaction does not create background levels of fluorescence that interfere with detection and measurement of the specific nucleic acid of the transgene that is being detected.

### EXAMPLE 2

Plant material obtained from Sigma Aldrich Chemical Company containing 5% Roundup Ready Soybeans, 5% Bt 176 maize and 5% Bt-11 maize have been analyzed for the presence of the specific transgenes which provide the insect resistance and herbicide resistance traits to commercial plant lines having these specific traits using the methods described in Example 1. The results of these detection and amplification experiments are present in FIG. 3. The sequences of the primer pairs used in the assays are given in Table 1 below. The results of these experiments demonstrate the general applicability of the methods of the present application to the identification of a variety of different target nucleic acid sequences in a sample to be analyzed for small amounts of those target sequences

TABLE 1

| | |
|---|---|
| Primer | Sequence |

| | | |
|---|---|---|
| P35S1-5/2-3 | 5′ att gat gtg ata tct cca ctg acg t 3′ | SEQ ID NO. 3 |
| | 5′ cct ctc caa atg aaa tga act tcc t 3′ | SEQ ID NO. 4 |
| P35S-cf-3/4 | 5′ cca cgt ctt caa agc aag tgg 3′ | SEQ ID NO. 5 |
| | 5′ tcc tct cca aat gaa atg aac ttc c 3′ | SEQ ID NO. 6 |
| P3SS1-5′/nos-3 | 5′ att gat gtg ata tct cca ctg acg t 3′ | SEQ ID NO. 7 |
| | 5′ tta tcc tag ttt gcg cgc ta 3′ | SEQ ID NO. 8 |
| Mg 1/2 | 5′ tat ctc cac tga cgt aag gga tga c 3′ | SEQ ID NO. 9 |
| | 5′ tgc cct ata aca cca aca tgt gct t 3′ | SEQ ID NO. 10 |
| Mg 3/4 | 5′ agt atc ctt cgc aag acc ctt cct c 3′ | SEQ ID NO. 1 |
| | 5′ gca ttc aga gaa acg tgg cag taa c 3′ | SEQ ID NO. 2 |
| Cd01/cr02 | 5′ cgg atg gtc ctt atg caa ttt tgt c 3′ | SEQ ID NO. 11 |
| | 5′ ctc tcg gcg tag att tgg tac a 3′ | SEQ ID NO. 12 |
| Pe01/cr02 | 5′ aga ttc ttc act ccg atg cag cct a 3′ | SEQ ID NO. 13 |
| | 5′ ctc tcg gcg tag att tgg tac a 3′ | SEQ ID NO. 12 |
| Cry1a2-5′t35S3-3′ | 5′ tgg aca aca aec caa aca tca a 3′ | SEQ ID NO. 14 |
| | 5′ tgg att ttg gtt tta gga att aga aa 3′ | SEQ ID NO. 15 |
| Cry1a4-5′/3-3′ | 5′ gga caa caa cca caa cat caa c 3′ | SEQ ID NO. 16 |
| | 5′ cga tgg ggg tgt aac cgg t 3′ | SEQ ID NO. 17 |
| Cry1a4-5′/4-3′ | 5′ gga caa caa cca caa cat caa c 3′ | SEQ ID NO. 16 |
| | 5′ gca cga act cgc taa gca g 3′ | SEQ ID NO. 18 |
| Cry1a1/2 | 5′ cgg ccc cga gtt cac ctt 3′ | SEQ ID NO. 19 |
| | 5′ ctg ctg ggg atg atg ttg ttg 3′ | SEQ ID NO. 20 |
| Cm01/cr02 | 5′ cac tac aaa tgc cat cat tgc gat a 3′ | SEQ ID NO. 21 |
| | 5′ ctc tcg gcg tag att tgg tac a 3′ | SEQ ID NO. 12 |
| Cm01/pa01 | 5′ cac tac aaa tgc cat cat tgc gat a 3′ | SEQ ID NO. 21 |
| | 5′ aga tca tca atc cac tct tgt ggt g 3′ | SEQ ID NO. 22 |
| Cm01/bar1-5′ | 5′ cac tac aaa tgc cat cat tgc gat a 3′ | SEQ ID NO. 21 |
| | 5′ gat agc gct ccc gca gac 3′ | SEQ ID NO. 23 |
| Adh1 1-5′/nos-ter2-3′ | 5′ gca ctg aat ttg tga acc c 3′ | SEQ ID NO. 24 |
| | 5′ cta tat ttt gtt ttc tat cgc 3′ | SEQ ID NO. 25 |
| Bar3-5′/1-3′ | 5′ cat cgt caa cca cta cat cga ga 3′ | SEQ ID NO. 26 |
| | 5′ gat agc gct ccc gca gac 3′ | SEQ ID NO. 27 |
| Bar2-5′/2-3′ | 5′ act ggg ctc cac gct cta ca 3′ | SEQ ID NO. 28 |
| | 5′ aaa ccc acg tca tgc cag ttc 3′ | SEQ ID NO. 29 |

1. A method of identifying the presence of a target nucleic acid in a sample wherein the target nucleic acid is replicated and said replication of the target is detected as the consumption of a deoxynucleotide triphosphate precursor, the method comprising:
adding an oligonucleotide primer which hybridizes to the sample target nucleic acid to the sample;
subjecting the sample DNA and priner to a polymerase reaction in the presence of a mixture of all dNTP's required for replication of the target nucleic acid whereby the deoxynucleotides will become incorporated and release pyrophosphate (PPi) proportional to the length of the DNA extension product; and
detecting any release of PPi enzymatically;
whereby any release of PPi is indicative of incorporation of deoxynucleotide or dideoxynucleotide and the presence of the target DNA.

2. A method as in claim 1, wherein the target nucleic acid is replicated in a reaction selected from the group consisting of a polymerase extension reaction, a polymerase chain reaction (PCR), a ligase chain reaction (LCR), a rolling circle replication reaction (RCR) and a nucleic acid sequence based amplification reaction (NASBA).

3. A method as in claim 2 wherein the target is replicated in a polymerase chain reaction.

4. A method as claimed in claim 1, wherein the release of PPi is detected by means of a Luciferase-Luciferin-based reaction.

5. A method as claimed in claim 1, wherein PPi release is detected using ATP sulfurylase and luciferase.

6. A method as claimed in claim 1, where the PPi detection enzymes are included in the polymerase reaction step and the polymerase reaction and PPi release detection steps are performed substantially simultaneously.

7. A method as claimed in claim 1, further comprising adding a dATP analogue which is capable of acting as a substrate for a polymerase, but incapable of acting as a substrate for a PPi detection enzyme.

8. A method as claimed in claim 7, wherein the dATP analogue is deoxyadenosine alpha thiotriphosphate

9. A method as claimed in claim 1, wherein the sample DNA or oligonucleotide primer is immobilized or provided with means for attachment to a solid support.

10. A method as claimed in claim 1, wherein exonuclease deficient high fidelity polymerase is used.

11. A method as in claim 1 wherein a heat resistant polymerase is used.

12. A method as claimed in claim 1, wherein the sample DNA is first amplified.

13. A method as claimed in claim 1, for use with a multiplicity of sample DNA sequences, wherein said DNA sequences are arranged in assay format on a solid surface.

14. The method according to claim 1 wherein said nucleic acid sample is obtained from a biological sample.

15. The method according to claim 1 wherein said target nucleic acid is a microbial or viral nucleic acid.

16. The method according to claim 15 wherein said target nucleic acid target is a viral nucleic acid.

17. The method according to claim 1 where said nucleic acid sample is obtained from a food source.

18. The method according to claim 17 wherein said food source is a plant.

19. The method according to claim 1 wherein said target nucleic acid contains a nucleic acid sequence non-native to the sample.

20. A kit for use in a method as defined in claim 1, comprising:
a polymerase;
detection enzyme means for identifying PPi release;
dNTP's, or optionally deoxynucleotide analogues, optionally including, in place of dATP, a dATP analogue which is capable of acting as a substrate for a polymerase but incapable of acting as a substrate for a said PPi-detection enzyme; and
optionally a target specific primer which hybridizes to the target DNA and is recognized as a primer by a polymerase, wherein the polymerase replicates the target DNA.

21. A kit as claimed in claim 20, wherein the detection enzyme means comprise a Luciferase-Luciferin-based enzymatic reaction.

22. A kit as claimed in claim 21, wherein the detection enzyme means comprise ATP sulfurylase and luciferase.
